(19) United States
(12) Patent Application Publication
(10) Pub. No.: US 2018/0192863 A1
(43) Pub. Date: Jul. 12, 2018

(54) METHOD FOR OBSERVING THE PARANASAL SINUS

(71) Applicant: OLYMPUS CORPORATION, Tokyo (JP)

(72) Inventor: Masahiro YOSHINO, Machida-shi (JP)

(73) Assignee: OLYMPUS CORPORATION, Tokyo (JP)

(21) Appl. No.: 15/402,297

(22) Filed: Jan. 10, 2017

(51) Int. Cl. A61B 1/233; A61B 1/06; A61B 90/30; A61B 1/00; A61B 34/20

(52) U.S. Cl. CPC A61B 1/233; A61B 1/0661; A61B 90/30; A61B 2090/363; A61B 34/20; A61B 2034/2055; A61B 2034/2068; A61B 1/00009

(57) ABSTRACT

A method for observing a paranasal sinus, includes, inserting an endoscope into the paranasal sinus, emitting light from the outside of a body after inserting the endoscope into the paranasal sinus so that a landmark is formed on an inner wall of the paranasal sinus, and operating the endoscope toward the periphery of the landmark so that at least part of the landmark is shown in an endoscopic image.

### 1. FIELD OF THE INVENTION

This invention relates to a method for observing a paranasal sinus whereby the inside of an observation target paranasal sinus can be visually recognized.

### 2. DESCRIPTION OF THE RELATED ART

For example, US2008/0097154A1 discloses an apparatus which serves to treat diseases of paranasal sinuses. As typified thereby, endoscopic surgical procedures are widespread in the treatment of chronic sinusitis.

### BRIEF SUMMARY OF THE INVENTION

A method for observing a paranasal sinus includes, inserting an endoscope into the paranasal sinus, emitting light from the outside of a body after inserting the endoscope into the paranasal sinus so that a landmark is formed on an inner wall of the paranasal sinus, and operating the endoscope toward the periphery of the landmark so that at least part of the landmark is shown in an endoscopic image.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing the overall configuration of an endoscopic system according to a first embodiment;

FIG. 2 is a side view through a part around a distal configuration portion of an endoscope insertion portion of the endoscopic system shown in FIG. 1;

FIG. 3 is a schematic diagram showing a state where a paranasal sinus is observed by the endoscopic system shown in FIG. 1, and showing the inside of the paranasal sinus in a broken manner;

FIG. 4 is a schematic diagram schematically showing a state where a landmark formed in the inner wall of the paranasal sinus is entirely or partly caught in a viewing field (endoscopic image) of an endoscope shown in FIG. 1; and

FIG. 5 is a schematic diagram schematically showing a state where the landmark is caught in the center of the viewing field (endoscopic image) of the endoscope shown in FIG. 4.

### DETAILED DESCRIPTION OF THE INVENTION

### Embodiment

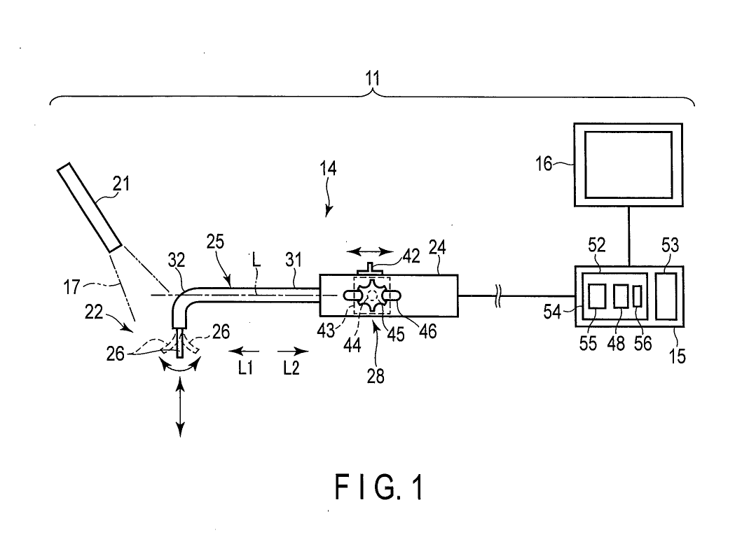

Hereinafter, an endoscopic system and a method for observing a paranasal sinus using the same will be described with reference to FIG. 1 to FIG. 5.

As shown in FIG. 1, an endoscopic system 11 has an insertion device 14 which is used to be inserted into, for example, a nasal cavity 12 and a paranasal sinus 13, a control unit 15 connected to the insertion device 14, a display unit 16 connected to the control unit 15, and a light source 21 which emits light 17 from the outside of the body to form a landmark 18. The insertion device 14 is provided separately from the display unit 16 and the control unit 15. The display unit 16 comprises a general liquid crystal monitor, and can display a figure acquired by an endoscope 22 as an image (endoscopic image 63).

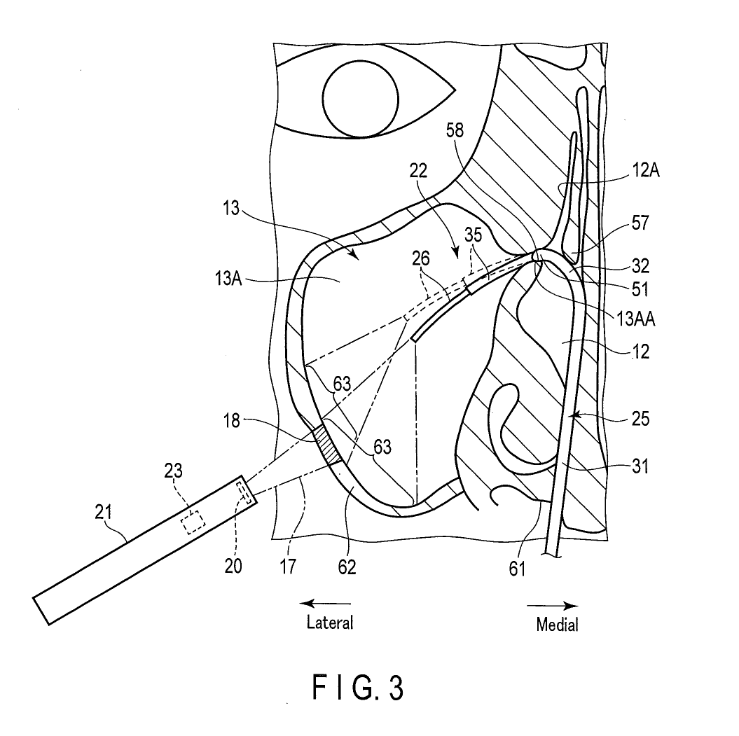

As the light source 21, an exclusive visible light lamp can be used, but a general small-sized flashlight may be used instead. The light source 21 has an LED light emitting element, but may include a filament electric bulb or may include an infrared lamp. The light source 21 may include a light emitting element (red LED) which includes much red light that is high in permeability. The light source 21 has a battery therein, but may be supplied with electric power from an external electric power supply. The light source 21 may have a control circuit 23 to intermittently emit light (to flash light) (see FIG. 3). The light source 21 may flash light many times in a short time so that user's attention can be easily drawn. The light source 21 may further have a switch (button) such that the user can suitably change over between a normal continuous on-state and the aforementioned intermittent flashing state. The light from the light source 21 is mostly visible light, but may include light having frequency bands other than those of the visible light, such as infrared light. The light source 21 may have a plate-shaped member 20 provided with a slit at a position right in front of the light emitting element (see FIG. 3). The landmark 18 can have various geometrical shapes by the passage of the light emitted from the light emitting element through the slit.

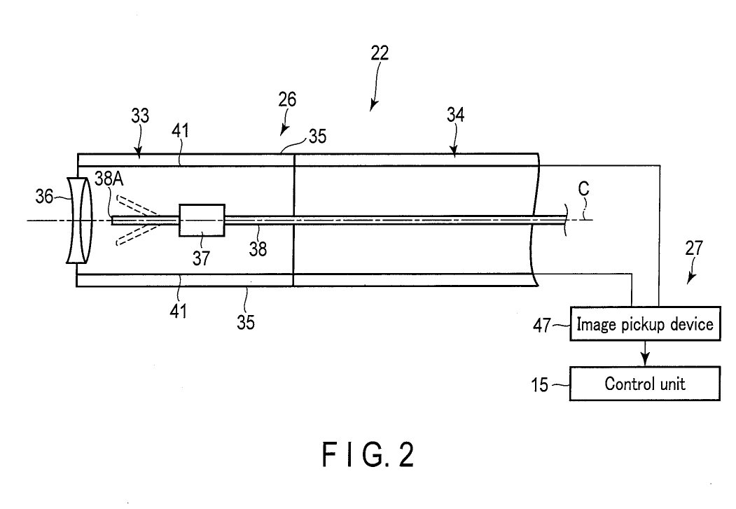

As shown in FIG. 1 and FIG. 2, the insertion device 14 has a grasp portion 24 which serves as a handle of the device and which also serves as an outer envelope, a cylindrical guide pipe 25 protruding from the grasp portion 24, an endoscope insertion portion 26 which is put through the guide pipe 25 and the grasp portion 24, an endoscopic imaging unit 27 (imaging unit) provided inside the grasp portion 24, and a curving operation portion 28 provided in the grasp portion 24. The grasp portion 24 is, for example, circularly cylindrical, and forms a part (housing) to be grasped by the hand of the user (surgeon).

Although the endoscope 22 has the endoscope insertion portion 26 and the endoscopic imaging unit 27 in the present embodiment as shown in FIG. 2, the endoscope 22 may have a configuration in which the endoscope insertion portion 26 and the endoscopic imaging unit 27 are integrated. Explanations are provided so that as shown in FIG. 1, L is the axial direction (central axis direction) of a later-described main body 31 of the guide pipe 25, L1 is a direction toward a later-described elbow portion 32 in the axial direction L, and L2 is a direction away from the elbow portion 32 (a direction toward the grasp portion 24).

The endoscope 22 comprises what is known as a scanning endoscope. The endoscope 22 (endoscope insertion portion 26) is configured to be flexible. Therefore, the endoscope insertion portion 26 can curve in accordance with the shape of the guide pipe 25 by being put through the guide pipe 25. The direction of the endoscope insertion, portion 26 can be adjusted by the guide pipe 25. As shown in FIG. 2, a central axis C is defined in the endoscope insertion portion 26 along its longitudinal direction. As shown in FIG. 1 and FIG. 3, the endoscope insertion portion 26 can move along the central axis C direction to protrude from the guide pipe 25.

As shown in FIG. 2, the endoscope insertion portion 26 has a distal configuration portion 33 located on the distal side in the central axis C direction, a flexible tube 34 provided closer to the proximal side in the central axis C direction than the distal configuration portion 33, a pair of wires (pull-wires) provided on the left and right over the distal end of a sheath 35 and the grasp portion 24, the cylindrical sheath 35 which covers the distal configuration portion 33, the flexible tube 34, and the wires, an illumination window 36, a rotation unit 37, an illumination fiber 38, and light receiving fibers 41. In the present embodiment, the sheath 35 is structured to be able to curve in a left-right direction (or an up-down direction) as shown in FIG. 1 together with the endoscope insertion portion 26 (the flexible tube 34) held inside the sheath 35.

As shown in FIG. 1, the grasp portion 24 may be further provided with a back-and-forth moving mechanism 42 which moves the endoscope insertion portion 26 back and forth relative to the guide pipe 25 in the axial direction L. The back-and-forth moving mechanism 42 comprises, for example, a knob portion which can move a support unit 43 back and forth. That is, in a medical examination, the surgeon who is the user can obtain a desired image of the inside of a sinus (e.g. the nasal cavity 12 and the paranasal sinus 13) of a medical examinee by changing the position of the endoscope insertion portion 26 using, for example, the back-and-forth moving mechanism 42 or by changing the curving angle of the endoscope insertion portion 26 as will be described later in a state where the endoscope insertion portion 26 is inserted in the sinus.

The curving operation portion 28 has the support unit 43 which is housed in the grasp portion 24 slidably in the axial direction L of the guide pipe 25, a shaft 44 which is rotatably supported on the support unit 43, a dial 45 (knob, rotation knob) which is fixed to one end of the shaft 44, and an unshown sprocket which is fixed to the other end of the shaft inside a case of the support unit 43. The shaft 44 protrudes to the outside of the grasp portion 24 from a long hole 46 formed in the grasp portion 24. A chain which can mesh with the sprocket is connected to the end of the wire. In response to the rotation of the dial 45, one of the pair of wires is pulled, and the other of the pair of wires loosens, so that the distal end of the sheath 35 is pulled, and the sheath 35 is curved to the left or right in FIG. 1. The sheath 35 curves so that the endoscope insertion portion 26 (the flexible tube 34) therein curves. The curving direction of the sheath 35 is one example, and it should be appreciated that the sheath 35 may curve to the far side and the near side in the plan of FIG. 1, or may be curvable in four upward, downward, leftward, and rightward directions. The wires (linear members) are pulled and can thereby adjust the curving angle of the endoscope 22 (the endoscope insertion portion 26).

As shown in FIG. 2, the illumination fiber 38 is optically connected to a second light source provided adjacent to the control unit 15. The light receiving fibers 41 are optically connected to an image pickup device 47. The distal ends of the light receiving fibers 41 are exposed to the outside in the vicinity of the distal configuration portion 33. Thus, the endoscope 22 can acquire a figure via the light receiving fibers 41 in the distal configuration portion 33.

As shown in FIG. 2, the endoscopic imaging unit 27 has the image pickup device 47 comprising, for example, a CCD or a CMOS. The image pickup device 47 comprises, for example, an element which can receive visible light and then convert the visible light into an electric signal. The image pickup device 47 may comprise a group of elements including, for example, the above element which can receive visible light and convert the visible light into an electric signal, and an element which can receive, for example, infrared light other than visible light and convert the infrared light into an electric signal. The endoscopic imaging unit 27 can acquire a figure obtained by the distal configuration portion 33 of the endoscope insertion portion 26. More specifically, the image pickup device 47 converts the light from the light receiving fibers 41 into an electric signal and then sends the electric signal to the control unit 15.

The rotation unit 37 is electrically connected to the control unit 15. The rotation unit 37 comprises, for example, a motor, and is, for example, spirally vibrated under the control of the control unit 15 (a motor driver 48). Thus, a distal end 38A of the illumination fiber 38 is spirally vibrated in accordance with the operation of the rotation unit 37. Therefore, the surface of a subject is spirally scanned with the illumination light from the illumination fiber 38 through the distal end 38A of the illumination fiber 38 and the illumination window 36. The light receiving fibers 41 receives return light from the subject, and then guides the light to the image pickup device 47. The image pickup device 47 sends the light received by the light receiving fibers 41 to the control unit 15 as the electric signal. The control unit 15 forms the electric signal into an image, suitably processes the image, and displays the image on the display unit 16.

As shown in FIG. 1, the guide pipe 25 is substantially “L”-shaped as a whole, and has a cylindrical (circularly cylindrical) shape which is bent partway into an elbow shape. The guide pipe 25 has the main body 31 attached at one end to the grasp portion, the elbow portion 32 provided at the other end of the main body 31, and a distal end 51 which protrudes from the elbow portion 32 in a direction different from the main body 31 (i.e. laterally).

The endoscope insertion portion 26 can be put through the guide pipe 25 (guide member). The inner wall of the guide pipe 25 can guide the endoscope insertion portion 26 which moves back and forth along the central axis C. The guide pipe 25 is preferably, for example, fixedly provided in the grasp portion 24, but may be rotatable relative to the grasp portion 24 around the axial direction L. In this case, the grasp portion 24 may be provided with a rotation knob to rotate the guide pipe 25 around the axial direction L.

The control unit 15 shown in FIG. 1 has, for example, a control unit main body 52 comprising a general computer, and an electric power supply 53 provided separately from the control unit main body 52. The control unit main body 52 comprises a housing 54, a circuit board 55 disposed in the housing 54, a CPU, a ROM, and a RAM that are mounted on the circuit board 55, an HDD 56 (hard disk drive), a software which is installed in the HDD 56 to perform various kinds of control on the insertion device 14, and a motor driver 48 which is disposed in the housing 54 to control the rotation unit 37. The electric power supply 53 can supply electric power to the rotation unit 37. The control unit 15 can perform, for example, the following control on each part of the insertion device 14. The control unit 15 can control the rotation unit 37 which swings the illumination fiber 38 via the motor driver 48, and thereby adjust, for example, the rotation number thereof. The control unit 15 can control the second light source to adjust a light amount to be supplied to the illumination fiber 38. The control unit 15 can process, into an image, the electric signal corresponding to the figure acquired by the image pickup device of the insertion device 14, and display this image (endoscopic image) on the display unit 16.

Now, a method for observing a paranasal sinus using the endoscopic system 11 according to the present embodiment is described with reference to FIG. 3 to FIG. 5. The explanation is provided on the assumption that the medical examinee is seated on a seat.

As shown in FIG. 3, the nasal cavity 12 is located substantially in the center of the face and extends in an anterior posterior direction. In FIG. 3, the anterior posterior direction is a direction that passes through the plan. A maxillary sinus 13A of the paranasal sinus 13 extends from the nasal cavity 12 in a lateral direction which is substantially 90° different from the anterior posterior direction. Between the nasal cavity 12 and the maxillary sinus 13A, a middle nasal concha 57 and an unciform 58 which are organs that separate the nasal cavity 12 from the maxillary sinus 13A are present. Therefore, the user pushes away the middle nasal concha 57 and the unciform 58 by the guide pipe 25, and at the same time brings the endoscope 22 inserted in the nasal cavity 12 to the vicinity of an opening 13AA of the maxillary sinus 13A to observe the inside of the maxillary sinus 13A. Further, the user needs to put the endoscope 22 into the opening 13AA of the maxillary sinus 13A to move the endoscope 22 forward toward the maxillary sinus 13A. Thus, the user needs to operate the endoscope 22 at an angle that deviates substantially 90° from the anterior posterior direction to observe the inside of the maxillary sinus 13A, and the user tends to lose his/her bearings during the observation of the maxillary sinus 13A and in its anterior posterior direction in the case of a conventional endoscope.

In the method for observing the paranasal sinus according to the present embodiment, the surgeon who is the user puts the guide pipe 25 into the nasal cavity 12 from an external nostril 61 of the medical examinee (subject) in a medical examination. In this instance, the guide pipe 25 and the endoscope 22 can move back and forth along the anterior posterior direction of the head. In this condition, the user can put the guide pipe 25 (the distal end 51, the elbow portion 32) into the space between a wall 12A which defines the surrounding of the external nostril 12 and the middle nasal concha 57 in a state where the distal end 51 of the guide pipe 25 faces, for example, downward in a vertical direction. In this way, the user can move the guide pipe 25 toward the posterior side of the head of the medical examinee while pushing away the middle nasal concha 57 by the elbow portion 32. FIG. 3 shows a state where the guide pipe 25 is put in the right external nostril 12 of the medical examinee.

When the distal end 51 of the guide pipe 25 has come close to the opening 13AA of the maxillary sinus 13A, the user twists the grasp portion 24 about 60° to 90° clockwise around the axial direction L so that the distal end 51 can climb over the unciform 58. Accordingly, the distal end 51 of the guide pipe 25 rotates around the axial direction L to face laterally from the downward side in the vertical direction. As a result, the distal end 51 faces laterally to climb over the unciform, and can confront the opening 13AA of the maxillary sinus 13A.

In this state, if necessary, the user finely adjusts the inclination of the endoscope 22 and the position of the distal configuration portion 33 by use of the image (endoscopic image) obtained by the endoscope 22. The user then protrudes the endoscope insertion portion 26 (the endoscope 22) from the distal end 51 of the guide pipe 25, and inserts the endoscope insertion portion 26 into the opening 13AA of the maxillary sinus 13A while checking the endoscopic image. The user further protrudes the endoscope insertion portion 26 from the guide pipe 25, and locates the distal end of the endoscope insertion portion 26 in the maxillary sinus 13A. The emission of the light 17 toward the paranasal sinus 13 from the outside of the body described below at the stage before the insertion of the endoscope 22 into the opening 13AA is not preferable because the position of the opening 13AA becomes unclear. That is, if the light 17 is emitted from the outside of the body, transmitted light that is thereby generated scatters around surrounding tissues other than the landmark 18, and the whole endoscopic image becomes reddish, so that the position of the opening 13AA becomes difficult to identify.

In a state where the distal end of the endoscope insertion portion 26 is located in the maxillary sinus 13A, the user or his/her assistant uses the light source 21 to emit the light 17 toward the position of the maxillary sinus 13A (the paranasal sinus 13) from the outside of the body of the medical examinee. Consequently, as shown in FIG. 3, the light 17 emitted from the light source 21 permeates through, for example, the skin of the medical examinee so that the landmark 18 which is bright as if spotlighted can be formed on an inner wall 62 of the maxillary sinus 13A. The landmark 18 is red or reddish, and is indicated brighter than the surrounding. It is also possible to form the shape of the landmark 18 into various shapes by projecting the light 17 emitted from the light emitting element on the living body through the slit. That is, the landmark 18 can have various geometrical shapes such as a circular shape, a quadrangular shape, a triangular shape, a polygonal shape, an arrow shape, and a star shape.

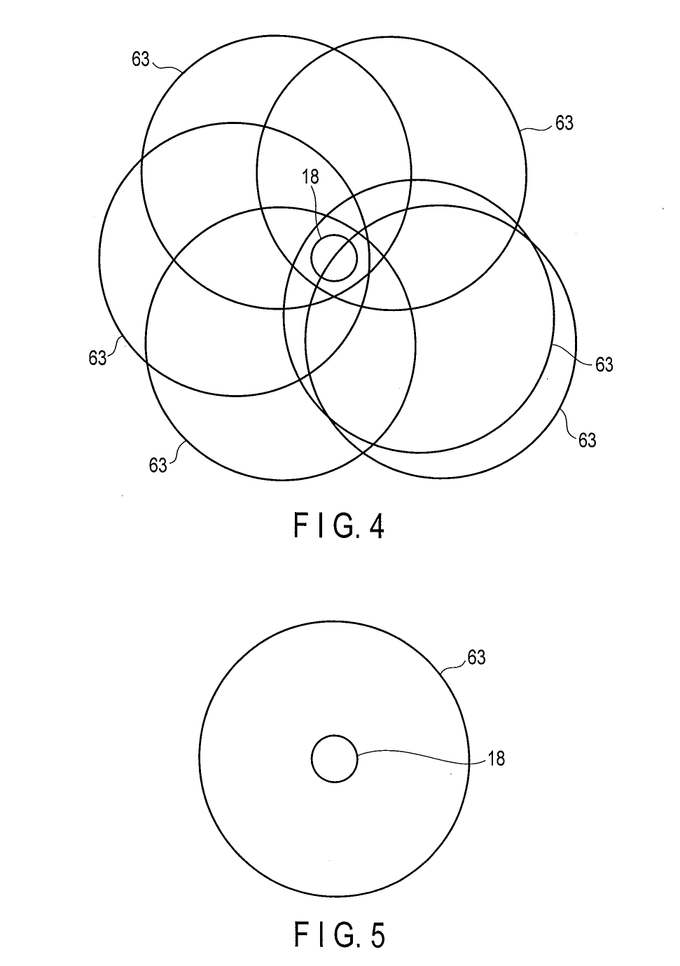

Among visible lights, light having a red frequency band is high in permeability, so that the landmark 18 is formed in red or in a reddish color. As shown in FIG. 4, the user can catch this landmark 18 in the endoscopic image 63. Various situations shown in FIG. 4 are possible as situations in which the landmark 18 is caught in the endoscopic image 63. In any situation, the landmark 18 has only to be entirely or partly caught in the endoscopic image 63 (the viewing field of the endoscope 22). In a state where the landmark 18 is caught in a part of the endoscopic image 63 in this way, the endoscope 22 can be operated toward the periphery of the landmark 18 (the observation direction of the endoscope 22 can be determined). The user may keep on the observation if the landmark 18 does not interfere with the observation (this is a first mode). In contrast, when the landmark 18 interferes with the observation, the user can turn off the light source 21 to eliminate the landmark 18 (this is a second mode). It is naturally also possible to return to the first mode from the second mode.

Furthermore, the light 17 from the light source 21 can be intermittently emitted under the control of the control circuit 23. This allows alternation between the first mode which forms the landmark 18 and which is suited to the recognition of the position in the paranasal sinus 13, and the second mode which eliminates the landmark 18 and which is suited to the observation in the paranasal sinus 13.

Alternatively, when the user needs to more precisely position the endoscope 22 in the paranasal sinus 13, the user may catch this landmark 18 in the center of the endoscopic image 63 as shown in FIG. 5. This permits more precise positioning of the endoscope 22. This also permits pinpoint observation of a predetermined position in the paranasal sinus 13. When the light 17 for the formation of the landmark 18 interferes with the observation even after the above positioning, the light source 21 can be turned off to eliminate the landmark 18. Moreover, even after such positioning, the light 17 can be intermittently emitted from the light source 21. This allows alternation between the first mode which forms the landmark 18 and which is suited to the recognition of the position in the paranasal sinus 13, and the second mode which eliminates the landmark 18 and which is suited to the observation in the paranasal sinus 13.

According to the embodiment, the following can be said: In the method for observing a paranasal sinus, the endoscope 22 is inserted into the paranasal sinus 13, the light 17 is emitted from the outside of the body after the insertion of the endoscope 22 into the paranasal sinus 13 so that a landmark is formed on the inner wall of the paranasal sinus, and the endoscope 22 is operated toward the periphery of the landmark 18 so that at least part of the landmark 18 is shown in the endoscopic image 63. In general, in a sinus such as the paranasal sinus 13 which has anatomically no landmark 18 or a small number of landmarks, the user (surgeon) tends to lose his/her bearings. In this situation, it is difficult for the user to know how to operate the endoscope 22 so that the place in the sinus to be observed can be observed. According to the configuration described above, the endoscope 22 can be operated toward the periphery of the landmark 18 by use of the landmark 18, so that the user can efficiently conduct an observation without losing his/her bearings. Even if the landmark 18 is temporarily formed as above, the medical examinee is not harmed, which is significantly advantageous to both the user (surgeon) and the medical examinee.

After the endoscope 22 is operated, the emission of the light 17 is stopped, and the paranasal sinus 13 is then observed. According to this configuration, the landmark 18 can be eliminated when the landmark 18 interferes with the observation, so that the user's convenience can be improved.

In the process of the formation of the landmark 18, the light 17 emitted from the outside of the body is intermittently emitted. According to this configuration, it is possible to alternate between the mode which forms the landmark 18 and which is suited to the recognition of the position in the paranasal sinus 13, and the mode which eliminates the landmark 18 and which is suited to the observation in the paranasal sinus 13. Consequently, the following usage is possible: for example, when the position needs to be again moved after a predetermined time of observation, the endoscope 22 can be moved with reference to the landmark 18, and an observation is again conducted after the completion of the movement. This can more significantly improve the user's convenience. Alternatively, user's attention can be drawn if the flashing time of the light 17 is shortened.

In the operation of the endoscope 22, the endoscope 22 is operated so that the landmark 18 is located in the center of the endoscopic image 63. According to this configuration, when the endoscope 22 needs to be more precisely positioned, this can be achieved in an extremely simple way. This permits pinpoint observation of a part to be observed, and the user's convenience can be significantly improved.

(First Modification)

Now, the endoscopic system 11 according to a first modification and a method for observing the paranasal sinus 13 using the same are described. Here, parts different from those in the above embodiment are primarily described, and the same parts as those in the above embodiment are neither shown nor described. The present modification is particularly advantageous to, for example, a condition in which the light 17 from the light source 21 fully reaches the inner wall of the paranasal sinus 13 (e.g. on the anterior side of the paranasal sinus 13).

In the present modification, the control unit 15 has two kinds of image generating modes: a first mode in which an image is formed so that the light 17 from the light source 21 to be the landmark 18 is included therein, and a second mode in which an image is formed without the light 17 from the light source 21 to be the landmark 18. In the first mode, among an R-signal, a G-signal, and a B-signal obtained from the image pickup device 47 of the endoscope 22, the R-signal (the frequency band of the light corresponding to the R-signal) alone is used to generate the endoscopic image 63. In the second mode, the endoscopic image 63 is generated from the G-signal (the frequency band of the light corresponding to the G-signal) and the B-signal (the frequency band of the light corresponding to the B-signal). The first mode and the second mode that can be changed over between as above are obtained by a program stored in the control unit 15 (the control unit main body 52). The changeover between the first mode and the second mode may be manually performed by the user operation. In this case, a changeover switch (button) is preferably provided in the control unit main body 52 or the grasp portion 24. The changeover from the first mode to the second mode and the changeover from the second mode to the first mode may be automatically performed after the elapse of a predetermined time by use of a timer function obtained by the program stored in the control unit main body 52.

The first mode and the second mode can naturally be also obtained in different ways. For example, in the first mode, the light caught by the endoscope 22 is filtered and acquired only as the R-signal in the image pickup device 47. Similarly, in the second mode, the light caught by the endoscope 22 is filtered and acquired only as the G-signal and the B-signal in the image pickup device 47. In these cases, the distal configuration portion 33 preferably has therein a filter which can independently cut off the R-signal, the G-signal, and the B-signal. The changeover between the first mode and the second mode in this case may be performed by the program stored in the control unit main body 52, or may be manually performed by the user operation.

Now, a method for observing the paranasal sinus 13 using the endoscopic system 11 according to the present embodiment is described. The process up to the insertion of the endoscope insertion portion 26 into the maxillary sinus 13A is similar to that in the embodiment described above. The user protrudes the endoscope insertion portion 26 from the guide pipe 25, and locates the distal end of the endoscope insertion portion 26 in the maxillary sinus 13A.

The user or his/her assistant uses the light source 21 to emit the light 17 toward the position of the maxillary sinus 13A (the paranasal sinus 13) from the outside of the body of the medical examinee. Consequently, as shown in FIG. 3, the landmark 18 which is bright as if spotlighted is formed on the inner wall 62 of the maxillary sinus 13A. The landmark 18 is red or reddish, and is indicated brighter than the surrounding. The landmark 18 can have various shapes as in the embodiment described above. In this instance, the endoscope 22 and the control unit 15 are set to the first mode suited to the catching of the landmark 18. That is, the light 17 from the light source 21 which is emitted from the outside of the body is dominated by red light high in permeability in the landmark 18 and therearound. Therefore, the user can rapidly catch the landmark 18 by the first mode in which an image is formed by the R-signal alone.

As in the embodiment described above, there are various situations shown in FIG. 4 as situations in which the landmark 18 is caught in the endoscopic image 63. In any situation, the landmark 18 has only to be entirely or partly caught in the endoscopic image 63 (the viewing field of the endoscope 22). When the landmark 18 is entirely or partly caught in the endoscopic image 63 in this way, the endoscope 22 can be operated toward the periphery of the landmark 18. In this state, the user operates the switch to change over from the first mode suited to the catching of the landmark 18 to the second mode suited to observation (or the control unit main body 52 automatically changes over from the first mode to the second mode by timer control).

In the second mode, the endoscopic image 63 is generated by the G-signal and the B-signal. Thus, it is possible to cut off the red light which is dominant in the landmark 18 and therearound and thus generate the endoscopic image 63. As a result, the light for the formation of the landmark 18 does not interfere with the observation, and the user can observe the inside of the paranasal sinus 13 (the maxillary sinus 13A) as usual. Even when the R-signal is cut off among the R-signal, the G-signal, and the B-signal and an image is then formed, it is possible to obtain satisfactory image quality without any problem in the observation. The user can suitably change over between the first mode and the second mode (or the control unit main body 52 can automatically change over every predetermined time by timer control), and it is naturally also possible to return to the first mode from the second mode.

Alternatively, when the user needs to more precisely position the endoscope 22 in the paranasal sinus 13, the user may set the endoscope 22 and the control unit 15 to the first mode, and catch this landmark 18 in the center of the endoscopic image 63 as shown in FIG. 5. This permits more precise positioning of the endoscope 22. If the user then manually changes over from the first mode to the second mode (or the control unit main body 52 automatically changes over from the first mode to the second mode by timer control), pinpoint observation of a predetermined position in the paranasal sinus 13 is possible.

It is naturally possible to suitably change over between the aspect of the first modification and the aspect of the embodiment described above on the basis of the operation from the user.

According to the present modification, in the method for observing the paranasal sinus 13, the endoscope 22 is operated in the first mode in which an image is formed so that the landmark 18 is included therein, and an image is formed without the landmark 18 to observe the paranasal sinus 13 in the second mode following the first mode. For example, when the landmark 18 is formed on the inner wall 62 of the paranasal sinus 13, the light of the landmark 18 may result in interference with the observation. According to this configuration, the landmark 18 is used in the first mode when the endoscope 22 is operated, and in the following second mode, the inside of the paranasal sinus 13 can be observed without the landmark 18. Consequently, this landmark 18 does not interfere with the observation, and user's convenience can be further improved.

In this case, in the first mode, the R-signal of the observation light obtained in the endoscope 22 is formed into an image, and in the second mode, the G-signal and the B-signal of the observation light obtained in the endoscope 22 are formed into an image. In general, the light which is projected on the inner wall 62 of the paranasal sinus 13 through the skin of the medical examinee is dominated by red light (light having the frequency band corresponding to red) which is high in permeability during the permeation through, for example, the skin. According to the configuration described above, red light is acquired in the first mode so that the landmark 18 can be efficiently searched for. Moreover, in the second mode, it is possible to efficiently and precisely observe the paranasal sinus 13 by excluding the influence of the red light which is dominant as described above. Further, according to the configuration described above, it is possible to change over between the first mode and the second mode in a simple manner without any modification on the side of the light source 21. Consequently, the configuration of the endoscope 22 which enables the method for observing the paranasal sinus 13 can be simpler, and manufacturing costs of the endoscope 22 can be reduced.

(Second Modification)

Now, the endoscopic system 11 according to a second modification and a method for observing the paranasal sinus using the same are described. Here, parts different from those in the above embodiment are primarily described, and the same parts as those in the above embodiment are neither shown nor described. The present modification is particularly advantageous to, for example, a condition in which the distance from the skin of the medical examinee to the paranasal sinus 13 is long (the far side of the paranasal sinus 13), it is difficult for the light 17 from the light source 21 to fully reach the inner wall 62 of the paranasal sinus 13, and the landmark 18 is not easily viewed by the endoscope 22.

In the present modification, the control unit 15 has two kinds of image generating modes: a first mode in which an image is formed so that the landmark 18 is highlighted, and a second mode in which an image is formed so that the highlight of the landmark is undone. In the first mode, the endoscopic image 63 is generated so that among the R-signal, the G-signal, and the B-signal obtained from the image pickup device 47 of the endoscope 22, the R-signal is highlighted. Specifically, the R-signal is enhanced to 110 to 300% compared to that before corrected, and the G-signal and the B-signal are left unchanged, and then the endoscopic image 63 is generated. Consequently, it is possible to generate the endoscopic image 63 in which the light of the landmark 18 mainly comprising red light is amplified.

In the second mode, the enhancement of the R-signal is undone, and as usual, the R-signal, the G-signal, and the B-signal are equally taken in to generate the endoscopic image 63. The first mode and the second mode that can be changed over between as above are obtained by the program stored in the control unit main body 52. The changeover between the first mode and the second mode may also be manually performed by the user operation. In this case, a changeover switch (button) is preferably provided in the control unit main body 52 or the grasp portion 24. The changeover from the first mode to the second mode and the changeover from the second mode to the first mode may be automatically performed after the elapse of a predetermined time by use of a timer function obtained by the program stored in the control unit main body 52.

Now, a method for observing the paranasal sinus using the endoscopic system 11 according to the present embodiment is described. The process up to the insertion of the endoscope insertion portion 26 into the maxillary sinus 13A is similar to that in the embodiment described above. The user protrudes the endoscope insertion portion 26 from the guide pipe 25, and locates the distal end of the endoscope insertion portion 26 in the maxillary sinus 13A.

The user or his/her assistant uses the light source 21 to emit the light 17 toward the position of the maxillary sinus 13A from the outside of the body of the medical examinee. Consequently, as shown in FIG. 3, the landmark 18 which is bright as if spotlighted is formed on the inner wall 62 of the maxillary sinus 13A. The landmark 18 can have various shapes as in the embodiment described above. The landmark 18 is red or reddish, and is indicated brighter than the surrounding. However, when the distance in which the light 17 emitted from the light source 21 permeates through, for example, the skin of the medical examinee is longer (e.g. when the far side of the paranasal sinus 13 is observed), the landmark 18 may not be bright enough.

In this instance, the endoscope 22 and the control unit main body 52 are set to the first mode suited to the catching of the landmark 18. That is, the light 17 from the light source 21 which is emitted from the outside of the body is dominated by red light high in permeability in the landmark 18 and therearound. Therefore, the user can rapidly recognize the landmark 18 and catch the landmark 18 in the endoscopic image 63 by enhancing the R-signal and then forming an image in the first mode.

As in the embodiment described above, there are various situations shown in FIG. 4 as situations in which the landmark 18 is caught in the endoscopic image 63. In any situation, the landmark 18 has only to be entirely or partly caught in the endoscopic image 63 (the viewing field of the endoscope 22). When the landmark 18 is entirely or partly caught in the endoscopic image 63, the endoscope 22 can be operated toward the periphery of the landmark 18. In this state, the user operates the switch to change over from the first mode suited to the catching of the landmark 18 to the second mode suited to observation (or the control unit main body 52 automatically changes over from the first mode to the second mode by timer control).

In the second mode, as usual, the R-signal, the G-signal, and the B-signal are equally acquired to generate the endoscopic image 63. In the case of the present modification, it is assumed that the light of the landmark 18 is originally weak, so that the endoscopic image 63 which has no problem in the observation can be generated even in a state where the landmark 18 is formed. As a result, the user can observe the inside of the paranasal sinus 13 (the maxillary sinus 13A) as usual. The user can suitably change over between the first mode and the second mode (or the control unit main body 52 can suitably change over every predetermined time by timer control), and it is naturally also possible to return to the first mode from the second mode.

Alternatively, when the user needs to more precisely position the endoscope 22 in the paranasal sinus 13, the user may set the endoscope 22 and the control unit 15 to the first mode, and catch this landmark 18 in the center of the endoscopic image 63 as shown in FIG. 5. This permits more precise positioning of the endoscope 22. If the user then manually changes over from the first mode to the second mode (or the control unit main body 52 automatically changes over from the first mode to the second mode by timer control), pinpoint observation of a predetermined position in the paranasal sinus 13 is possible.

It is naturally possible to suitably change over between the aspect of the second modification, the aspect of the embodiment described above, and the aspect of the first modification on the basis of the operation from the user.

According to the present modification, in the method for observing the paranasal sinus, the endoscope 22 is operated in the first mode in which an image is formed so that the landmark 18 is highlighted, and an image is formed so that the highlight is undone to observe the paranasal sinus 13 in the second mode following the first mode. According to this configuration, for example, when the light of the landmark 18 is weak in the case of the observation of the far side of the paranasal sinus 13, the position of the landmark 18 can be more easily recognized. This can further improve the user's convenience.

In this case, in the first mode, an image is formed so that the R-signal of the observation light obtained in the endoscope 22 is highlighted. According to this configuration, the most dominant red light in the light 17 which permeates through, for example, the skin of the medical examinee and is then projected on the inner wall 62 of the paranasal sinus 13 is efficiently acquired so that the endoscopic image 63 can be generated. As a result, it is possible to enhance the landmark 18 in the endoscopic image 63 in a simple manner. Thus, the position of the landmark 18 can be more easily recognized, and it is possible to prevent the structure of the endoscopic system 11 used in this method for observing the paranasal sinus from being complicated. This permits the reduction of manufacturing costs of the endoscopic system 11 used in this method.

While the embodiment and each of the modifications have been described in detail with reference to the drawings, so far, this invention is not limited to the embodiment described above, and the components can be modified without departing from the spirit of the invention. Although the right maxillary sinus 13A of the medical examinee (subject) is observed in FIG. 3, it is also naturally possible to observe the left maxillary sinus 13A. Although the maxillary sinus 13A of the paranasal sinus 13 is observed in the cases described by way of example in the embodiment and each of the modifications described above, it is also naturally possible to apply the above method for observing the paranasal sinus to the observations of the frontal sinus and the ethmoidal sinus of the paranasal sinus 13. Although it is assumed that the medical examinee is seated on the seat in the embodiment and each of the modifications, the present invention is naturally also applicable to the case in which the medical examinee is horizontally lying.

Furthermore, although the paranasal sinus 13 is targeted for observation in the embodiment and each of the modifications described above, it is also possible to apply the above observation method to sinuses and tubes in human bodies such as bladders in which the number of landmarks is small as in the paranasal sinus 13.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method for observing a paranasal sinus, comprising:
inserting an endoscope into the paranasal sinus;
emitting light from the outside of a body after inserting the endoscope into the paranasal sinus so that a landmark is formed on an inner wall of the paranasal sinus; and
operating the endoscope toward the periphery of the landmark so that at least part of the landmark is shown in an endoscopic image.

2. The method for observing the paranasal sinus according to claim 1, wherein after the endoscope is operated, the light emitted from the outside of the body is stopped, and the paranasal sinus is then observed.

3. The method for observing the paranasal sinus according to claim 1, wherein the endoscope is operated in a first mode in which an image is formed so that the landmark is included therein, and
an image is formed without the landmark to observe the paranasal sinus in a second mode following the first mode.

4. The method for observing the paranasal sinus according to claim 3, wherein in the first mode, an R-signal of observation light obtained in the endoscope is formed into an image, and
in the second mode, a G-signal and a B-signal of the observation light obtained in the endoscope are formed into an image.

5. The method for observing the paranasal sinus according to claim 1, wherein the endoscope is operated in a first mode in which an image is formed so that the landmark is highlighted, and
an image is formed so that the highlight is undone to observe the paranasal sinus in a second mode following the first mode.

6. The method for observing the paranasal sinus according to claim 5, wherein in the first mode, an image is formed so that an R-signal of observation light obtained in the endoscope is highlighted.

7. The method for observing the paranasal sinus according to claim 1, wherein in the process of the formation of the landmark, the light emitted from the outside of the body is intermittently emitted.

8. The method for observing the paranasal sinus according to claim 1, wherein the endoscope is operated so that the landmark is located in the center of the endoscopic image.
